(19) United States
(12) Patent Application Publication
(10) Pub. No.: US 2026/0146987 A1
(43) Pub. Date: May 28, 2026

(54) METHOD AND SYSTEM FOR ESTIMATING ORGANIC CARBON CONTENT OF WETLAND SOIL WITH DIFFERENT RESTORATION YEARS

(71) Applicants: Northeast Institute of Geography and Agroecology, Chinese Academy of Sciences, Changchun City (CN); CHANGCHUN NORMAL UNIVERSITY, Changchun City (CN)

(72) Inventors: Geng CUI, Changchun City (CN); Yan LIU, Changchun City (CN); Weiyu HUANG, Changchun City (CN); Shouzheng TONG, Changchun City (CN); Yu AN, Changchun City (CN); Mingye ZHANG, Changchun City (CN)

(73) Assignees: Northeast Institute of Geography and Agroecology, Chinese Academy of Sciences, Changchun City (CN); CHANGCHUN NORMAL UNIVERSITY, Changchun City (CN)

(21) Appl. No.: 19/401,226

(22) Filed: Nov. 25, 2025

(30) Foreign Application Priority Data
Nov. 25, 2024 (CN) 202411691933.X

(51) Int. Cl. G01N 33/24; G06V 10/26; G06V 10/54; G06V 10/58; G06V 20/13

(52) U.S. Cl. CPC G01N 33/24; G06T 11/10; G06V 10/26; G06V 10/54; G06V 10/58; G06V 20/13

(57) ABSTRACT

A method for estimating the organic carbon content of wetland soil with different restoration years, which relates to the technical field of carbon sink accounting. The method includes: setting a plurality of sampling points in a target research area, and collecting soil samples; preprocessing satellite image data and determining first input features, determining second input features according to geographic coordinates of the sampling points, and inputting the features into a regression prediction model to obtain a calculation result; generating soil organic matter content data distribution according to the calculation result; and dividing sample areas with different restoration years according to land use history or restoration measure records by using space-for-time substitution, and counting the soil organic matter content and total amount in each sample area according to the soil organic matter content data distribution to determine the organic carbon content of soil with different restoration years.

### INCORPORATION BY REFERENCE

This application claims priority to China Patent Application No. 202411691933.X filed November 25 2024, the contents of which are hereby incorporated by reference in their entirety.

### TECHNICAL FIELD

The present disclosure relates to the technical field of wetland carbon sink accounting, and in particular, to a method and system for estimating the organic carbon content of wetland soil with different restoration years.

### BACKGROUND

Wetlands are a huge organic carbon pools in terrestrial ecosystems and an important part of the Earth's carbon cycle system. Wetlands account for only 5% to 8% of the total land area, but they are one of the world's largest soil carbon pools. The organic carbon reserve in wetlands is about 455 to 700 Pg (1 Pg=1 Gt=1 billion tons), accounting for 20% to 30% of the total surface carbon reserve in the terrestrial ecosystem, and its carbon reserve per unit area ranks first among all types of terrestrial ecosystems, and is three times that of forest ecosystems.

The acceleration of global warming and human development and utilization have led to the shrinkage of wetland area, the deterioration of wetland ecological environment and changes in wetland carbon cycle process. With the proposal of “dual carbon” goals in China, a series of important ecological restoration projects have been carried out to maintain the stability of carbon sinks in wetlands and restore the carbon sink function of degraded wetlands. How to monito and account carbon sinks in important wetlands and evaluate the role of major ecological restoration projects in enhancing the carbon sink function of wetland ecosystems has become an urgent problem to be solved. However, there is currently a lack of dynamic monitoring of carbon sinks after the restoration of important wetlands, and the understanding of the synergistic effect between wetland ecological restoration projects and carbon sequestration is not comprehensive enough. There is an urgent need to carry out monitoring and accounting of carbon sinks in important wetland restoration areas.

Due to the distinct regional and complex distribution of wetland resources, which include various types and dynamic changes, current research on wetland carbon measurement models and the accuracy of results still face many challenges. Numerous researchers have calculated the carbon reserves and the amount of carbon sinks of wetlands in different research areas in China. However, due to differences in wetland types, geographical conditions, and calculation methods, the results exhibit significant uncertainty. The main reasons are as follows: firstly, there is a lack of long-term data, the wetland restoration process spans a long period of time, and it is difficult to continuously monitor the rate of organic carbon accumulation over different years, resulting in a lack of long-term trend data, which in turn affects the accurate analysis of the relationships between restoration years and organic carbon reserves. Secondly, the spatial heterogeneity has an impact, and due to significant differences in hydrological conditions, vegetation types and soil composition, wetland ecosystems have different carbon accumulation characteristics in different areas, making it challenging to establish a unified carbon reserve estimation method across a wide area. Thirdly, due to the difficulty in sample acquisition and complex wetland environments, the collection of deep soil samples is particularly limited by technologies and equipment, making it difficult to obtain accurate organic carbon content data. The differences in organic carbon content in soil at different depths also interfere with accurate estimation. Therefore, how to accurately and efficiently calculate the soil organic carbon content in wetland restoration areas is a current research hotspot and challenge.

### SUMMARY

In order to overcome the shortcomings of the existing technologies, an objective of the present disclosure is to provide a method and system for estimating the organic carbon content of wetland soil with different restoration years. By integrating remote sensing data, geospatial information and soil sampling data and performing machine learning, the organic carbon content of wetland soil with different restoration years can be accurately estimated, thereby solving the problems of lack of data, time and labor consumption, and limited accuracy in traditional methods for evaluating the carbon reserves in wetland ecosystems.

To achieve the above objective, the present disclosure provides the following scheme.

A method for estimating the organic carbon content of wetland soil with different restoration years includes:
setting a plurality of sampling points in a target research area, and collecting soil samples at the sampling points;
preprocessing satellite image data in the target research area to obtain preprocessed remote sensing image data;
determining first input features according to the preprocessed remote sensing image data, and determining second input features according to geographic coordinates of the sampling points; the first input features including: index bands, a radar band index, a spectral band index, and a texture feature image; the second input features including feature values of the sampling points;
inputting the first input features and the second input features into a well-trained regression prediction model for soil organic matter content to obtain a calculation result;
generating soil organic matter content data distribution according to the calculation result; and
dividing sample areas with different restoration years according to land use history or restoration measure records by using space-for-time substitution, and counting the soil organic matter content and total amount in each sample area according to the soil organic matter content data distribution to determine the organic carbon content of soil with different restoration years.

Preferably, the soil sample has a depth of 0 to 30 cm.

Preferably, the satellite image data includes: Sentinel-1 and Sentinel-2 data.

Preferably, the method for preprocessing satellite image data of the target research area includes: cloud removal and denoising.

Preferably, determining the first input features according to the preprocessed remote sensing image data includes:
determining a gray-level co-occurrence matrix according to the preprocessed remote sensing image data;
performing a band extraction according to the preprocessed remote sensing image data to obtain the index bands;
performing a band index extraction according to the preprocessed remote sensing image data to obtain the radar band index and the spectral band index;
Performing a feature extraction according to the gray-level co-occurrence matrix to obtain texture features; and
generating the texture feature image according to the texture features.

Preferably, the index bands include: a normalized difference vegetation index (NDVI), an enhanced vegetation index (EVI), and a near infrared (NIR) band index.

Preferably, the regression prediction model for soil organic matter content is obtained by training a random forest regression model.

Preferably, an evaluation index for the random forest regression model includes: a determination coefficient and a root mean square error.

The present disclosure further provides a system for estimating the organic carbon content of wetland soil with different restoration years, which includes:
a sampling module configured to set a plurality of sampling points in a target research area, and collecting soil samples at the sampling points;
an image preprocessing module configured to preprocess satellite image data in the target research area to obtain preprocessed remote sensing image data;
a feature determination module configured to determine first input features according to the preprocessed remote sensing image data, and to determine second input features according to geographic coordinates of the sampling points; the first input features including index bands, a radar band index, a spectral band index, and a texture feature image; the second input features including feature values of the sampling points;
a machine learning module configured to input the first input features and the second input features into a well-trained regression prediction model for soil organic matter content to obtain a calculation result;
a data distribution generation module configured to generate soil organic matter content data distribution according to the calculation result; and
an estimation restoration module configured to divide sample areas with different restoration years according to land use history or restoration measure records by using space-for-time substitution, and count the soil organic matter content and total amount in each sample area according to the soil organic matter content data distribution to determine the organic carbon content of soil with different restoration years.

According to the specific embodiments provided by the present disclosure, the present disclosure discloses the following technical effects.

The present disclosure provides a method for estimating the organic carbon content of wetland soils with different restoration years, which includes: setting a plurality of sampling points in a target research area, and collecting soil samples at the sampling points; preprocessing satellite image data in the target research area to obtain preprocessed remote sensing image data; determining first input features according to the preprocessed remote sensing image data, and determining second input features according to geographic coordinates of the sampling points; the first input features including index bands, a radar band index, a spectral band index, and a texture feature image; the second input features including feature values of the sampling points; inputting the first input features and the second input features into a well-trained regression prediction model for soil organic matter content to obtain a calculation result; generating soil organic matter content data distribution according to the calculation result; and dividing sample areas with different restoration years according to land use history or restoration measure records by using space-for-time substitution, and counting the soil organic matter content and total amount in each sample area according to the soil organic matter content data distribution to determine the organic carbon content of soil with different restoration years. According to the method and systems of the present disclosure, a remote sensing technology and a machine learning algorithm are used to achieve rapid and non-destructive estimation of organic carbon content in wetland soils, thereby reducing the workload and cost of field sampling.

### BRIEF DESCRIPTION OF DRAWINGS

In order to more clearly illustrate the technical solutions in the embodiments of the present disclosure or the existing technologies, the accompanying drawings used in the embodiments will be briefly introduced below. Obviously, the accompanying drawings described below are only some embodiments of the present disclosure. For a person of ordinary skill in the art, other drawings can be obtained based on these accompanying drawings without creative effort.

FIG. 1 is a flowchart of a method provided by an embodiment of the present disclosure;

FIG. 2 is a schematic diagram of a modeling process of an SOC random forest regression model provided by an embodiment of the present disclosure;

FIG. 3 is a distribution diagram of organic carbon content provided by an embodiment of the present disclosure; and

FIG. 4 is a schematic diagram of the organic carbon content in 0 -30 cm of restored plots with different years provided by an embodiment of the present disclosure.

### DETAILED DESCRIPTION OF EMBODIMENTS

The technical solutions of the present disclosure will be clearly and completely described below with reference to the accompanying drawings of the embodiments of the present disclosure. Obviously, the described embodiments are only some embodiments of the present disclosure, but not all embodiments. Based on the embodiments of the present disclosure, all other embodiments obtained by a person of ordinary skill in the art without creative effort are within the protection scope of the present disclosure.

An objective of the present disclosure is to provide a method for estimating the organic carbon content of wetland soil with different restoration years. A remote sensing technology and a machine learning algorithm are used to achieve rapid and non-destructive estimation of the organic carbon content of wetland soil, thereby reducing the workload and cost of field sampling.

In order to make the above-mentioned objective, features and advantages of the present disclosure more apparent and understandable, the present disclosure will be further described in detail below with reference to the accompanying drawings and specific embodiments.

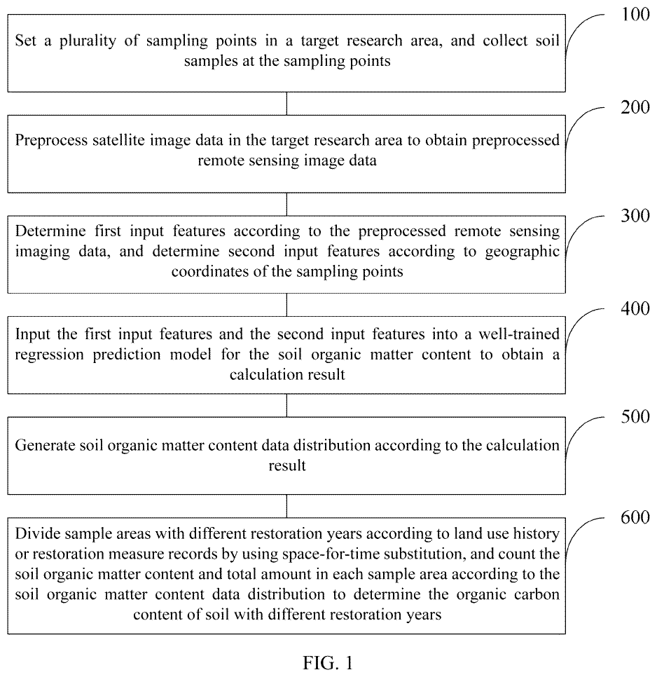

FIG. 1 is a flowchart of the method provided by an embodiment of the present disclosure. As shown in FIG. 1, the present disclosure provides a method for estimating the organic carbon content of wetland soil with different restoration years, which includes:
step 100: setting a plurality of sampling points in a target research area, and collecting soil samples at the sampling points;
step 200: preprocessing satellite image data in the target research area to obtain preprocessed remote sensing image data;
step 300: determining first input features according to the preprocessed remote sensing image data, and determining second input features according to geographic coordinates of the sampling points; the first input features including: index bands, a radar band index, a spectral band index, and a texture feature image; the second input features including feature values of the sampling points;
step 400: inputting the first input features and the second input features into a well-trained regression prediction model for soil organic matter content to obtain a calculation result;
step 500: generating soil organic matter content data distribution according to the calculation result; and
step 600: dividing sample areas with different restoration years according to land use history or restoration measure records by using space-for-time substitution, and counting the soil organic matter content and total amount in each sample area according to the soil organic matter content data distribution to determine the organic carbon content of soil with different restoration years.

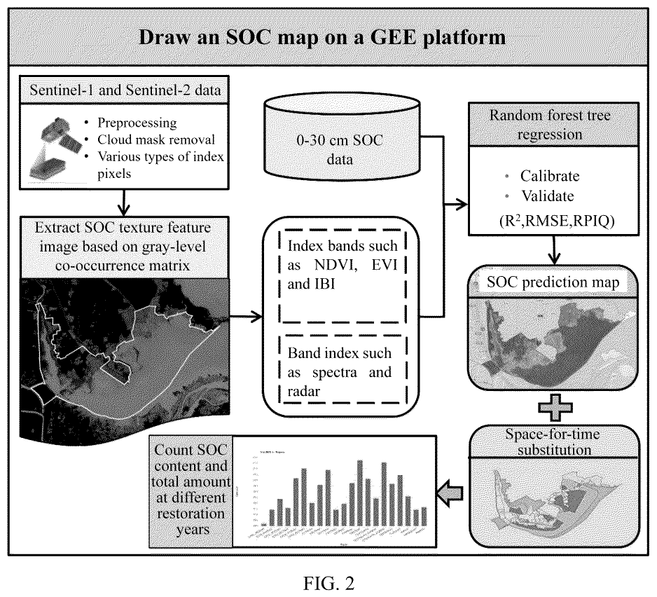

Specifically, as shown in FIG. 2, according to the present disclosure, by integrating remote sensing data, geospatial information and soil sampling data, a random forest regression model is constructed for machine learning, the organic carbon content in wetland soil with different restoration years can be estimated accurately, and the problems of the lack of data, time and labor consumption and limited accuracy in carbon reserve estimation of wetland ecosystems can be solved. The specific implementation steps are as follows.

I. Field sampling and testing: soil samples at 0 -30 cm are collected in the target research area, with one sample point per 16.5 square kilometers.

In the target research area, a soil auger with a fixed volume (V) is used in this embodiment to collect soil samples, mainly for determining soil organic carbon, etc. On each soil sampling plot, the samples are collected at 0 -10 cm, 10-20 cm, and 20-30 cm. A soil auger with an inner diameter of ≥5 cm is used to collect samples. More than 200 g of mixed samples are collected from each layer. After non-soil materials are removed, the samples are aliquoted into self-sealing bags, labeled, and brought back to a laboratory, and the soil samples with natural horizons reserved are air-dried. The number of the collected sample points should be one point per 16.5 square kilometers, and the latitudes and longitudes of the sampling points are recorded.

II. Selection and processing of satellite impact data: Sentinel-1 and Sentinel-2 data, including optical and radar images, are used. The preprocessing step includes cloud removal and denoising to clear raw image data. Various types of index images, such as a normalized difference vegetation index (NDVI), an enhanced vegetation index (EVI), and a near infrared (NIR) band index, are generated.

In this embodiment, satellite remote sensing data of Sentinel-1 and Sentinel-2 are acquired from relevant data sources such as the official website of the European Space Agency (ESA). It ensures that the data covers the research area and an appropriate time period is selected to acquire representative surface information. The acquired data is then subjected to geometric correction, atmospheric correction, radiometric correction, and topographic correction to remove the influences of the atmosphere on surface reflectivity and other factors, thereby improving the accuracy of the data. Meanwhile, cloud detection bands in Sentinel-2 data are used to generate a cloud mask image, and pixels obscured by clouds are removed to ensure the accuracy of subsequent analysis.

III. Extraction of texture feature images and determination of feature values of sampling points: based on the preprocessed remote sensing image data, various spectral indices, radar indices and texture features of wetland surfaces are extracted from biophysical parameters by using the gray-level co-occurrence matrix (GLCM) method. At the same time, the feature values of each sampling point are acquired according to geographic coordinates of the soil sampling points.

In this embodiment, specific bands (e.g., red-edge bands and near-infrared bands) in the Sentinel-2 data are selected to calculate the gray-level co-occurrence matrix, and texture features such as contrast, energy, homogeneity and correlation are extracted from the gray-level co-occurrence matrix. An SOC texture feature image is generated according to the texture features. Extraction of other index bands mainly includes: calculating NDVI, EVI, and NIR. These indices can reflect information such as vegetation growth, soil moisture, and ground coverage, which helps in the prediction of SOC. Finally, radar band indices such as radar backscattering coefficients are extracted from the Sentinel-1 data and combined with spectral bands in the Sentinel-2 data to construct a multi-source dataset. At the same time, the feature values of each sampling point are acquired according to the geographical coordinates of the soil sampling points.

IV. Model construction and training: a random forest regression model is used to train and validate the measured soil organic carbon content to determine the model accuracy.

In this embodiment, SOC texture feature images, index bands such as NDVI, EVI, and NIR, as well as spectral and radar band indices are used as input features. At the same time, a random forest algorithm is used to establish the regression prediction model for SOC content. The model performances are optimized by adjusting parameters of the random forest (e.g., the number and maximum depth of trees). The model is then validated using an independent validation dataset. The model parameters are adjusted according to the validation result to improve the prediction accuracy of the model. Finally, methods such as cross-validation may be used to further evaluate the stability and generalization ability of the model.

V. Acquisition of organic carbon content of soil with different years by space-for-time substitution: a spatiotemporal distribution map of organic carbon in wetland soil is generated based on a validated random forest regression model, and the organic carbon content in soil with different years is determined by using space-for-time substitution.

In this embodiment, the optimized random forest model is applied to the dataset of the entire research area. The SOC content data distribution is generated according to a model calculation result, and the data is visualized to show spatial distribution characteristics of SOC content. Sample areas with different restoration years are divided according to land use history or restoration measure records by using space-for-time substitution, and the soil organic matter content and total amount are counted in each sample area to determine the organic carbon content in soil with different restoration years.

As an optional embodiment, an assessment for carbon sequestration and enhancement benefits in a restoration area of returning fishery to wetness is carried out in the Sihong Hongze Lake Wetland National Nature Reserve (hereinafter referred to as Hongze Lake Reserve) in Jiangsu Province. To track changes in carbon reserves over the years within the restoration area, experiments are conducted to estimate the organic carbon content of wetland soil with different restoration years based on a random forest regression model, and the research is carried out using the following methods.

I. Field sampling and testing: within the target research area, a soil auger with a fixed volume (V) is used to collect soil samples, mainly for determining soil organic carbon, etc. On each sampling plot, samples are collected at depths of 0 -10 cm, 10-20 cm, and 20-30 cm. A soil auger with an inner diameter of ≥5 cm is used to collect samples. More than 200 g of mixed samples are collected from each layer. After non-soil materials are removed, the samples are aliquoted into self-sealing bags, labeled, and brought back to a laboratory, and the soil samples with natural horizons reserved are air-dried. A total of 80 points are sampled throughout the area to determine the soil organic carbon content.

II. Selection and processing of satellite impact data: various biophysical parameters, including NDVI and EVI, as well as texture feature images extracted by GLCM are extracted as input features of the random forest regression model by using Sentinel-1 and Sentinel-2 satellite data.

III. Extraction of texture feature images and determination of feature values of sampling points:
(1) input data: Sentinel-1 and Sentinel-2 data (including optical and radar images) are used. The preprocessing step includes cloud removal and denoising to clear raw image data. Various types of index images, such as NDVI, EVI and NIR, are generated; and
(2) extraction of feature values according to step (1): texture feature images are extracted from biophysical parameters by using the GLCM method, and the feature values for each sampling point are acquired according to the geographical coordinates of the soil sampling points.

IV. Model construction and training: based on the stratification of field soil samples, the organic carbon content of the soil layer at 0-0.3 m is used as a target value. The random forest regression model is used for training. Through calibration and validation processes, relevant evaluation indices are calculated. A determination coefficient (R²) reaches 0.75 and a root mean square error (RMSE) is 5.5, indicating that the model simulation effect is relatively good.

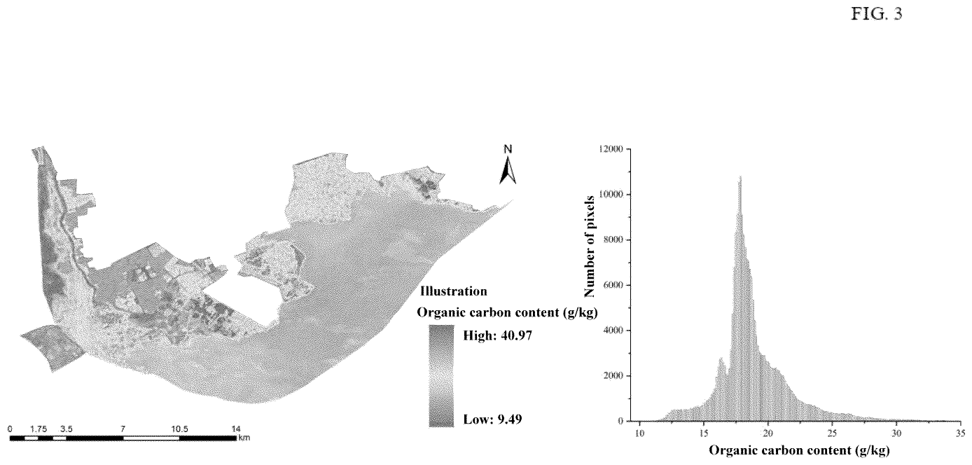

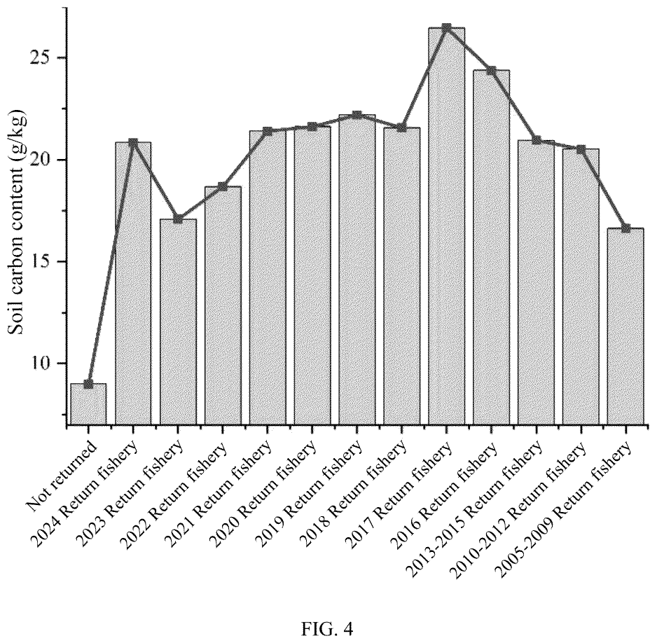

V. Acquisition of organic carbon content of soil with different years by space-for-time substitution: based on the random forest model, the soil organic carbon content in the soil layer at 0 -30 cm in the Hongze Lake Nature Reserve is estimated (FIG. 3). The results show that the organic carbon content is as high as 40.97 g/kg and as low as 9.49g/kg. The organic carbon content in soil in more than 80% of the area is between 16 g/kg and 21 g/kg. The grids of the soil organic carbon content result are subjected to flood inundation extraction by using spatial vector data of the restoration area to obtain the average organic carbon content of the soil in areas with different restoration years (FIG. 4). The average organic carbon content of plots in the Hongze Lake Nature Reserve with different restoration years show a trend of first increasing and then decreasing with the increase of restoration years. The highest organic content is found in a restored plot in 2017 (26.46 g/kg), followed by a plot in 2016 (24.37 g/kg), and the lowest is found in an unrestored plot (8.99 g/kg).

Corresponding to the above method, as shown in FIG. 2, the present disclosure further provides a system for estimating the organic carbon content of wetland soil with different restoration years, which includes:
a sampling module configured to set a plurality of sampling points in a target research area and collect soil samples at the sampling points;
an image preprocessing module configured to preprocess satellite image data in the target research area to obtain preprocessed remote sensing image data;
a feature determination module configured to determine first input features according to the preprocessed remote sensing image data, and to determine second input features according to geographic coordinates of the sampling points; the first input features including: index bands, a radar band index, a spectral band index, and a texture feature image; the second input features including feature values of the sampling points;
a machine learning module configured to input the first input features and the second input features into a well-trained regression prediction model for soil organic matter content to obtain a calculation result;
a data distribution generation module configured to generate soil organic matter content data distribution according to the calculation result; and
a restoration estimation module configured to divide sample areas with different restoration years according to land use history or restoration measure records by using space-for-time substitution, and counting the soil organic matter content and total amount in each sample area according to the soil organic matter content data distribution to determine the organic carbon content of soil with different restoration years.

The beneficial effects of the present disclosure are as follows.
(1) The present disclosure integrates Sentinel-1 and Sentinel-2 satellite data, and through preprocessing and cloud removal masking, high-resolution optical and radar data are effectively utilized to enhance the information accuracy, richness and accuracy of soil organic matter content (SOC) prediction.
(2) The present disclosure constructs an efficient random forest regression model, which can effectively process a large number of nonlinear relationships and complex feature interactions, reduce the risk of overfitting, and improve the prediction accuracy and generalization ability of SOC content, thereby achieving accurate estimation of wetland soil organic carbon content.
(3) The present disclosure is not only applicable to the estimation of organic carbon content in wetland soil with different restoration years, but can also be extended to the assessment of carbon reserves in other types of ecosystems.
(4) The present disclosure achieves rapid and non-destructive estimation for organic carbon content in wetland soil by using a remote sensing technology and a machine learning algorithm, thereby reducing the workload and cost of field sampling.

The various embodiments in this specification are described in a progressive manner. Each embodiment focuses on the differences from other embodiments. The same or similar parts between the various embodiments can be referred to each other. The system disclosed in the embodiments is described in a relatively simple manner because it corresponds to the method disclosed in the embodiments. For relevant details, please refer to the method section.

Specific examples have been used herein to illustrate the principles and embodiments of the present disclosure. The above description of the embodiments is only for the purpose of helping to understand the method and core ideas of the present disclosure. At the same time, a person of ordinary skill in the art will recognize that, based on the ideas of the present disclosure, there will be changes in the specific embodiments and application scopes. In summary, the content of this specification should not be construed as a limitation of the present disclosure.

What is claimed is:

1. A method for estimating the organic carbon content of wetland soil with different restoration years, comprising:
setting a plurality of sampling points in a target research area, and collecting soil samples at the sampling points;
preprocessing satellite image data in the target research area to obtain preprocessed remote sensing image data;
determining first input features according to the preprocessed remote sensing image data, and determining second input features according to geographic coordinates of the sampling points, the first input features comprising: index bands, a radar band index, a spectral band index, and a texture feature image; the second input features comprising feature values of the sampling points;
inputting the first input features and the second input features into a well-trained regression prediction model for the soil organic matter content to obtain a calculation result;
generating soil organic matter content data distribution according to the calculation result; and
dividing sample areas with different restoration years according to land use history or restoration measure records by using space-for-time substitution, and counting the soil organic matter content and total amount in each sample area according to the soil organic matter content data distribution to determine the organic carbon content of soil with different restoration years, wherein determining the first input features according to the preprocessed remote sensing image data comprises:
determining a gray-level co-occurrence matrix according to the preprocessed remote sensing image data;
performing a band extraction according to the preprocessed remote sensing image data to obtain the index bands;
performing a band index extraction according to the preprocessed remote sensing image data to obtain the radar band index and the spectral band index;
performing a feature extraction according to the gray-level co-occurrence matrix to obtain texture features; and
generating the texture feature image according to the texture features.

2. The method for estimating the organic carbon content of wetland soil with different restoration years according to claim 1, wherein the soil sample has a depth of 0 -30 cm.

3. The method for estimating the organic carbon content of wetland soil with different restoration years according to claim 1, wherein the satellite image data comprises: Sentinel-1 and Sentinel-2 data.

4. The method for estimating the organic carbon content of wetland soil with different restoration years according to claim 1, wherein preprocessing the satellite image data of the target research area comprises: cloud removal and denoising.

5. The method for estimating the organic carbon content of wetland soil with different restoration years according to claim 1, wherein the index bands comprise: a normalized difference vegetation index (NDVI), an enhanced vegetation index (EVI), and a near infrared (NIR) band index.

6. The method for estimating the organic carbon content of wetland soil with different restoration years according to claim 1, wherein the regression prediction model for the soil organic matter content is obtained by training a random forest regression model.

7. The method for estimating the organic carbon content of wetland soil with different restoration years according to claim 6, wherein an evaluation index for the random forest regression model comprises a determination coefficient and a root mean square error.

8. A system for estimating the organic carbon content of wetland soil with different restoration years, comprising:
a sampling module configured to set a plurality of sampling points in a target research area and collecting soil samples at the sampling points;
an image preprocessing module configured to preprocess satellite image data in the target research area to obtain preprocessed remote sensing image data;
a feature determination module configured to determine first input features according to the preprocessed remote sensing image data, and to determine second input features according to geographic coordinates of the sampling points; the first input features comprising index bands, a radar band index, a spectral band index, and a texture feature image; the second input features comprising feature values of the sampling points;
a machine learning module configured to input the first input features and the second input features into a well-trained regression prediction model for soil organic matter content to obtain a calculation result;
a data distribution generation module configured to generate soil organic matter content data distribution according to the calculation result;
a restoration estimation module configured to divide sample areas with different restoration years according to land use history or restoration measure records by using space-for-time substitution, and count the soil organic matter content and total amount in each sample area according to the soil organic matter content data distribution to determine the organic carbon content of soil with different restoration years, wherein
determining the first input features according to the preprocessed remote sensing image data comprises:
determining a gray-level co-occurrence matrix according to the preprocessed remote sensing image data;
performing a band extraction according to the preprocessed remote sensing image data to obtain the index bands;
performing a band index extraction according to the preprocessed remote sensing image data to obtain the radar band index and the spectral band index;
performing a feature extraction according to the gray-level co-occurrence matrix to obtain texture features; and
generating the texture feature image according to the texture features.
